(19) United States
(12) Patent Application Publication
(10) Pub. No.: US 2022/0139508 A1
(43) Pub. Date: May 5, 2022

(54) COOL - A SCREENING COLLABORATIVE OPEN OUTCOMES TOOL

(71) Applicant: International Business Machines Corporation, Armonk, NY (US)

(72) Inventors: Mohamed Ghalwash, Yorktown Heights, NY (US); Vibha Anand, Cambridge, MA (US); Eileen Koski, New York, NY (US)

(21) Appl. No.: 17/090,499

(22) Filed: Nov. 5, 2020

(51) Int. Cl. G16H 10/60; G06F 16/2455; C12Q 1/6883

(52) U.S. Cl. CPC G16H 10/60; C12Q 2600/158; C12Q 1/6883; G06F 16/24556

(57) ABSTRACT

Techniques facilitating autoimmune disorder screening schedule evaluations. In one example, a system can comprise a processor that executes computer executable components stored in memory. The computer executable components can comprise a pre-processing component and an evaluation component. The pre-processing component can generate a biomarker dataset for a subpopulation using an aggregated database of biomarker data for a population comprising the subpopulation. The evaluation component can determine a performance metric for a screening schedule based on the biomarker dataset. The performance metric can quantify an effectiveness of the screening schedule in identifying subjects within the subpopulation that are at risk of developing an autoimmune disorder.

### BACKGROUND

The subject disclosure relates to computing devices, and more specifically, to techniques of facilitating autoimmune disorder screening schedule evaluations.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, systems, devices, computer-implemented methods, and/or computer program products that can facilitate evaluations of screening schedules for autoimmune disorders are described.

According to an embodiment, a system can comprise a processor that executes computer executable components stored in memory. The computer executable components can comprise a pre-processing component and an evaluation component. The pre-processing component can generate a biomarker dataset for a subpopulation using an aggregated database of biomarker data for a population comprising the subpopulation. The evaluation component can determine a performance metric for a screening schedule based on the biomarker dataset. The performance metric can quantify an effectiveness of the screening schedule in identifying subjects within the subpopulation that are at risk of developing an autoimmune disorder.

According to another embodiment, a computer-implemented method can comprise generating, by a system operatively coupled to a processor, a biomarker dataset for a subpopulation using an aggregated database of biomarker data for a population comprising the subpopulation. The computer-implemented method can further comprise determining, by the system, a performance metric for a screening schedule based on the biomarker dataset. The performance metric can quantify an effectiveness of the screening schedule in identifying subjects within the subpopulation that are at risk of developing an autoimmune disorder.

According to another embodiment, a computer program product can comprise a computer readable storage medium having program instructions embodied therewith. The program instructions are executable by a processor to cause the processor to perform operations. The operations can include generating, by the processor, a biomarker dataset for a subpopulation using an aggregated database of biomarker data for a population comprising the subpopulation. The operations can further include determining, by the processor, a performance metric for a screening schedule based on the biomarker dataset. The performance metric can quantify an effectiveness of the screening schedule in identifying subjects within the subpopulation that are at risk of developing an autoimmune disorder.

### DESCRIPTION OF THE DRAWINGS

FIG. 1 illustrates a block diagram of an example, non-limiting system that can facilitate evaluations of potential screening schedules for autoimmune disorders, in accordance with one or more embodiments described herein.

FIG. 2 illustrates an example, non-limiting operational flow for implementing an autoimmune disorder screening schedule evaluation process, in accordance with one or more embodiments described herein.

FIG. 3 illustrates an example, non-limiting operational flow for implementing an aggregated database registration process, in accordance with one or more embodiments described herein.

FIG. 4 illustrates an example, non-limiting operational flow for implementing a subpopulation configuration process, in accordance with one or more embodiments described herein.

FIG. 5 illustrates an example, non-limiting operational flow for implementing a screening schedule definition process, in accordance with one or more embodiments described herein.

FIG. 6 illustrates an example, non-limiting operational flow for implementing a screening schedule evaluation subprocess, in accordance with one or more embodiments described herein.

FIG. 7 illustrates an example, non-limiting operational flow for implementing a time-dependent distribution data subprocess, in accordance with one or more embodiments described herein.

FIG. 8 illustrates an example, non-limiting operational flow for implementing a screening schedule comparison subprocess, in accordance with one or more embodiments described herein.

FIG. 9 illustrates an, non-limiting graphical user interface for facilitating autoimmune disorder screening schedule evaluations, in accordance with one or more embodiments described herein.

FIG. 10 illustrates a flow diagram of an example, non-limiting computer-implemented method of facilitating autoimmune disorder screening schedule evaluations, in accordance with one or more embodiments described herein.

FIG. 11 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Type 1 diabetes (T1D) is a complex, heterogeneous, autoimmune disorder in which insulin-producing beta cells of the pancreas are mistakenly destroyed by the body's immune system. Research has indicated that T1D has both genetic and familial components and while often diagnosed early in life, can also emerge in adulthood. Patients with T1D remain insulin dependent for life and are at high risk for serious long-term complications such as heart and renal disease and diabetic retinopathy. For unknown reasons, T1D incidence rates have been rising dramatically, particularly among children below age 5, making research on prevention and early detection increasingly critical.

There is currently no cure or prevention for T1D and the etiology of the disease is not yet fully understood. Newly diagnosed patients often present with diabetic ketoacidosis (DKA), a life-threatening condition with potentially long-term consequences, so there is compelling need for improved prediction and prevention. The National Institutes of Health (NIH)-funded “The Environmental Determinants of Diabetes in the Young” (TEDDY) study is investigating environmental determinants of T1D and interventional trials (e.g., interventional trials performed by the TrialNET consortium) of therapeutic agents are focusing on delaying onset. Methodological improvements to patient identification via early screening or risk stratification for clinical trial recruitment can make important contributions to such research.

Even though the incidence of T1D is generally low, the incidence rate is increasing dramatically worldwide. Due to the lack of a cure or effective prevention, there is currently no broad-based screening for T1D. There have, however, been several large, longitudinal studies of children considered to be at risk—specifically children with first-degree relatives with T1D and/or with biochemical markers of T1D, specifically development of autoantibodies associated with T1D. Early identification of children at risk could help reduce the incidence of DKA at onset, which can be life-threatening, via improved monitoring of relevant changes in biomarkers and education. Perhaps most promising, there have been recent trials of agents that may delay onset, raising the hopes that screening can identify children at risk both for improved monitoring, as well as potential prophylactic treatments.

Moreover, while disease onset in purely genetic conditions is a given (certainty that the disease exists and will manifest, even if it is hard or impossible to predict when), in complex conditions (where gene and environmental interactions are thought to modulate disease processes) this is not the case. In complex conditions such as T1D, it is therefore very important to know the predictive value of developing the disease based on a test (screening test), in combination with other patient characteristics, and in a given time period (e.g. 10-years or 15-years in case of T1D). This may facilitate preventive interventions (research ongoing in this area).

Because of the multi-factorial nature of these complex conditions, the composition of affected subpopulations can also change over time. That variance in the composition of affected subpopulations creates a dynamic system in which to understand the predictive value or other performance metrics such as sensitivity. These metrics can help clinicians adjust practice patterns and policy makers inform population health decisions.

Embodiments of the disclosed framework for facilitating autoimmune disorder screening schedule evaluations offers researchers a tool to help them estimate levels and predictors of risk in study populations, by exploring the contribution of different risk factors to observed outcomes. Ultimately, this tool could be used by clinicians to evaluate the level of risk in their own patient populations to better assess appropriate measures, and characteristics that could be used to identify specific individuals at risk to enable improved education, monitoring or eligibility for clinical prophylaxis trials. In addition to T1D, there are other auto-immune disorders, such as Celiac Disease, where the same general conditions apply and where this tool could prove useful in both research and clinical care.

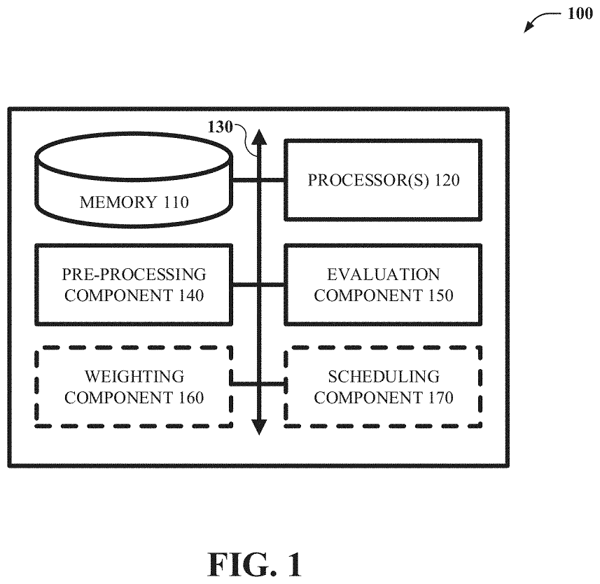

FIG. 1 illustrates a block diagram of an example, non-limiting system 100 that can facilitate evaluations of potential screening schedules for autoimmune disorders, in accordance with one or more embodiments described herein. System 100 includes memory 110 for storing computer-executable components and one or more processors 120 operably coupled via one or more communication busses 130 to memory 110 for executing the computer-executable components stored in memory 110. As shown in FIG. 1, the computer-executable components can include: pre-processing component 140; and evaluation component 150.

Pre-processing component 140 can generate a biomarker dataset for a subpopulation using an aggregated database of biomarker data for a population comprising the subpopulation. In an embodiment, the aggregated database can include: electronic health record data, disease registry data, or a combination thereof. In an embodiment, the aggregated database can include biomarker data in a non-standardized format. In an embodiment, pre-processing component 140 can generate the biomarker dataset by converting the biomarker data in the non-standardized format into a standardized format of the biomarker dataset. In an embodiment, pre-processing component 140 can generate the biomarker dataset by comparing metadata of the aggregated database with a filtering criterion that defines a distinguishing characteristic of the subpopulation. In an embodiment, pre-processing component 140 can generate an additional biomarker dataset for an additional subpopulation that is distinct from the subpopulation by virtue of a distinguishing characteristic.

Evaluation component 150 can determine a performance metric for a screening schedule based on the biomarker dataset. In an embodiment, the performance metric can quantify an effectiveness of the screening schedule in identifying subjects within the subpopulation that are at risk of developing an autoimmune disorder. In an embodiment, the performance metric can include: a specificity metric, a sensitivity metric, a positive predictive value metric, a negative predictive value metric, or a combination thereof. In an embodiment, the performance metric facilitates identifying a best screening schedule in terms of effectiveness in identifying at-risk subjects within the subpopulation based on the specific characteristics of the autoimmune disorder, as well as the specific characteristics of the subpopulation. In an embodiment, the performance metric can assist researchers, clinicians, and/or policy makers in carefully balancing the costs and risks associated with different potential screening schedules. For example, if screening is done too frequently, costs will be higher and patients may stop testing prematurely if results have all been negative; while if not done frequently enough, conditions may not be detected in time to intervene. In an embodiment, evaluation component 150 can generate time-dependent distribution data for a plurality of groups comprising the subpopulation using the screening schedule. In an embodiment, evaluation component 150 can generate comparison data for a plurality of screening schedules by analyzing respective performance metrics of the plurality of screening schedules determined using the biomarker dataset. In an embodiment, evaluation component 150 can determine one or more performance metrics for the screening schedule corresponding to an additional subpopulation based on an additional biomarker dataset.

In an embodiment, the computer-executable components stored in memory 110 can further include weighting component 160. Weighting component 160 can assign weights to subject-specific subsets of the biomarker dataset based on longitudinally available data to compensate for irregular data within the aggregated database. By way of example, in a gold standard research setting of a randomized, blinded clinical trial all patients enter a study with strictly specified inclusion or exclusion criteria that can be evaluated using specialized testing. Data (e.g., biomarker data) from the randomized, blinded trial is generally collected using case-report forms that involve uniform: data elements; data collection; and data formats. All patients entering the study can be followed at pre-specified intervals, so most data elements are collected at reasonable comparable time windows (e.g., every 3-months±4 weeks). Depending on a defined purpose of the study, all patients can be followed for either a uniform period of time, or until a specified endpoint occurs (e.g., death or disease onset).

Even in the gold standard research setting, some patients can be “lost to follow-up” before a target study end (or endpoint) for a number of reasons, such as moving out of a study area, having an intervening medical problem that triggers their removal from the study, or the patient simply stops coming for follow-up for unknown reasons. Sample size calculations for clinical trials typically take into account a number of patients likely to drop out or cross over (e.g., from control to treatment due to disease progression) so that enough patients will remain to draw valid conclusions about the study.

Embodiments of the present disclosure utilize aggregated databases of biomarker data comprising electronic health record data, disease registry data, and the like to evaluate screening schedules. Stated differently, embodiments of the present disclosure can evaluate screening schedules using data generated on the basis of routine clinical care—not with data generated from patients carefully tested, selected, and enrolled into the gold standard research setting of a randomized, blinded clinical trial. As such, embodiments of the present disclosure can evaluate screening schedules using real-world-evidence (or real-world-data) representing irregular data in that variable follow-up intervals, missing data and drop-outs are generally more prevalent in real-world-data than in data collected in the gold standard research setting of a randomized, blinded clinical trial.

For example, in routine clinical care, patients may change providers periodically due to relocation or changes in insurance coverage. As a result, patients may only occur in a given aggregated database for limited periods of time, as opposed to the length of time generally needed by a clinical trial to draw valid conclusions. Moreover, the frequency and regularity of clinical visits is generally less predictable in routine care than in a clinical trial. Therefore, important measurements do not generally occur among patients during clinical visits at routine, common time points, and there may be irregular gaps of varying length between expected clinical visits for a variety of reasons.

When dealing with rare or low prevalent conditions (e.g., T1D), this can make it extremely difficult, if not completely impossible, to identify sufficient numbers of patients with the necessary characteristics and sufficient follow-up data to evaluate screening schedules (e.g., should individuals be tested every XX months). Weighting component 160 facilitates the use of such irregular data in evaluating screening schedules by assigning weights to subject-specific subsets of a biomarker dataset based on longitudinally available data. In an embodiment, the assigned weights facilitate inclusion of biomarker data associated with all eligible patients by weighting the amount of follow-up available differentially such that a patient with very little follow-up data will have less impact on a screening schedule evaluation than a patient with more follow-up data. In an embodiment, weighting component 160 can assign weights using an inverse probability censoring weighting mechanism.

In an embodiment, the computer-executable components stored in memory 110 can further include scheduling component 170. Scheduling component 170 can create an optimal screening schedule for the subpopulation based on the biomarker dataset. In an embodiment, scheduling component 170 creates the optimal screening schedule using an objective function that maximizes a performance metric of a screening schedule by modifying one or more parameters of a screening schedule configuration based on the biomarker dataset. In an embodiment, scheduling component 170 can implement one or more machine learned model(s) trained to create an optimal screening schedule for a subpopulation based on a biomarker dataset. Any known artificial intelligence, machine learning, knowledge-based, or rule-based mechanisms can be used to train the one or more machine learned models using training data. Examples of such mechanisms include support vector machines, neural networks, expert systems, Bayesian belief networks, fuzzy logic, data fusion engines, classifiers, and the like. The training data can be obtained from data sets comprising historical biomarker data. The functionality of the computer-executable components utilized by the embodiments will be covered in greater detail below.

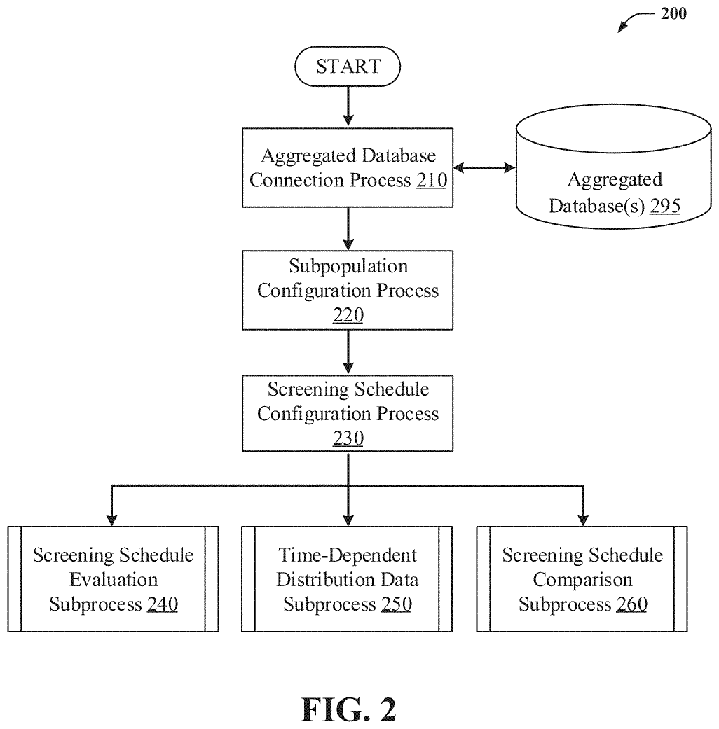

FIG. 2 illustrates an example, non-limiting operational flow for implementing an autoimmune disorder screening schedule evaluation process 200, in accordance with one or more embodiments described herein. At 210, system 100 can connect with one or more aggregated databases 295 of biomarker data for a population of patients (or subjects). As described in greater detail below, biomarker data sourced from the one or more aggregated databases 295 can facilitate evaluating screening schedules for autoimmune disorders. In an embodiment, the one or more aggregated databases 295 comprise metadata regarding patient characteristics associated with the biomarker data. In an embodiment, the metadata can facilitate ordering operations performed on biomarker data stored in a given aggregated database. Examples of such ordering operations include: sorting operations, filtering operations, grouping operations, clustering operations, and the like.

In an embodiment, the one or more aggregated databases 295 can include electronic health record data. As used herein, “electronic health record data” is clinical data associated with routine and/or acute care provided to patients of a medical practice. Electronic health record data can include medical and/or treatment histories of patients, such as family medical history, diagnoses, medications, treatment plans, immunization dates, allergies, radiology images, laboratory results, test results, and the like. In an embodiment, the medical practice can include: a solo medical practice, a group medical practice, and/or an employed physician practice.

In an embodiment, the one or more aggregated database(s) 295 can include disease registry data. As used herein, “disease registry data” is a collection of information about people who have a specific disease or medical condition. Examples of aggregated databases including disease registry data that are suitable for implementing the one or more aggregated databases 295 comprise: the Autoimmune Research Network (ARNet) database, the United States Immunodeficiency Network (USIDNET) Registry for Patients with Primary Immunodeficiency Diseases database, the Diabetes Collaborative Registry database, and the like.

At 220, system 100 generates a subpopulation configuration that defines a particular subpopulation (or subset of a population of patients) associated with biomarker data stored in aggregated database 295. At 230, system 100 generates a screening schedule configuration for evaluation by evaluation component 150. As used herein, a “screening schedule” is a timetable for administration of a screening test or a sequence of screening tests to detect potential health disorders or diseases in patients who do not exhibit any symptoms. In an embodiment, a screening test can be based on one or more biomarkers. At 240, evaluation component 150 of system 100 can determine a performance metric for a screening schedule based on a biomarker dataset for a subpopulation. At 250, evaluation component 150 can generate time-dependent distribution data for a plurality of groups comprising the subpopulation using the screening schedule. At 260, evaluation component 150 can generate comparison data for a plurality of screening schedules by analyzing respective performance metrics of the plurality of screening schedules determined using the biomarker dataset.

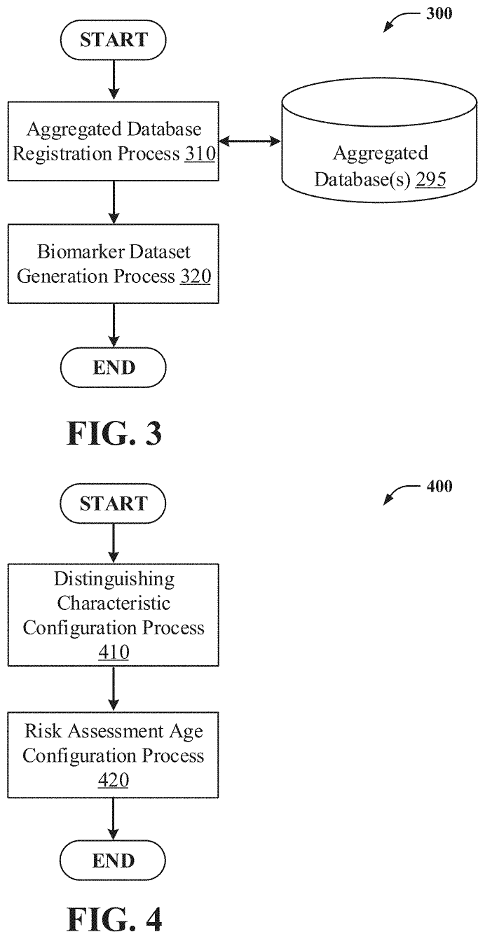

FIG. 3 illustrates an example, non-limiting operational flow for implementing an aggregated database registration process 300, in accordance with one or more embodiments described herein. In an embodiment, the aggregated database connection process 210 of autoimmune disorder screening schedule evaluation process 200 can be implemented using aggregated database registration process 300. At block 310, system 100 can register one or more aggregated databases 295 comprising biomarker data for a population of patients (or subjects) and/or metadata regarding patient characteristics associated with the biomarker data. In an embodiment, registering the one or more aggregated databases 295 can include storing registration data in memory (e.g., memory 110). Examples of such registration data include: a database name, address information (e.g., an Internet Protocol (IP) address), versioning information, security information (e.g., login credentials), and the like. In an embodiment, the registration data can be used to establish a connection between system 100 and the one or more aggregated databases 295.

At 320, pre-processing component 140 of system 100 can generate a biomarker dataset for a subpopulation (or subset of a population of patients) associated with biomarker data stored in the one or more aggregated databases 295. In an embodiment, pre-processing component 140 can generate the biomarker dataset by comparing metadata of the one or more aggregated databases 295 with a filtering criterion that defines a distinguishing characteristic of the subpopulation. In an embodiment, pre-processing component 140 can obtain the distinguishing characteristic from a subpopulation configuration stored in memory (e.g., memory 110). For example, a filtering criterion can define male as a distinguishing characteristic of the subpopulation. In this example, pre-processing component 140 can compare that filtering criterion with metadata the one or more aggregated databases 295 to identify a subset of the biomarker data that corresponds with male patients (or subjects). Pre-processing component 140 can utilize that subset of the biomarker data to generate a biomarker dataset for the subpopulation defined by the filtering criterion.

One skilled in the art will appreciate that a subpopulation configuration can include multiple distinguishing characteristics that define a particular subpopulation. By way of example, a subpopulation configuration can include female as a first distinguishing characteristic and below 10 years of age as a second distinguishing characteristics. In this example, pre-processing component 140 can compare filtering criteria based on the first and second distinguishing characteristics with metadata of the one or more aggregated databases 295 to identify a subset of the biomarker data that corresponds with female patients (or subjects) that are less than 10 years of age. Pre-processing component 140 can utilize that subset of the biomarker data to generate a biomarker dataset for the subpopulation defined by those filtering criteria.

In an embodiment, the one or more aggregated databases 295 can include biomarker data in a non-standardized format. By way of example, the one or more aggregated databases 295 can include a first aggregated database corresponding to a first clinical practice and a second aggregated database corresponding to a second clinical practice. In this example, the first and second clinical practices can utilize different hardware and/or software platforms to store data associated with routine clinical and/or acute care provided to patients, such as biomarker data and associated metadata. Depending on the particular hardware and/or software platform that the first and second clinical practices utilize, the first aggregated database can store biomarker data in a first format that is incompatible with a second format that the second aggregated database utilizes to store biomarker data.

In an embodiment, pre-processing component 140 can generate a biomarker dataset by converting the biomarker data in the non-standardized format into a standardized format of the biomarker dataset. Continuing with the example above in which the first and second aggregated databases store biomarker data in first and second formats, respectively, pre-processing component 140 can generate biomarker datasets in a third format that is incompatible with one or more of the first and second formats. To facilitate the utilization of biomarker data from incompatible sources (i.e., the first and second databases) in evaluating screening schedules, pre-processing component 140 can convert biomarker data in a non-standardized format (e.g., biomarker data in the first and/or second formats) into a standardized format (i.e., the third format) to generate biomarker datasets.

FIG. 4 illustrates an example, non-limiting operational flow for implementing a subpopulation configuration process 400, in accordance with one or more embodiments described herein. In an embodiment, the subpopulation configuration process 220 of autoimmune disorder screening schedule evaluation process 200 can be implemented using subpopulation configuration process 400. At block 410, system 100 can populate a subpopulation configuration with one or more distinguishing characteristics received from an entity. The one or more distinguishing characteristics can define a particular subpopulation (or subset of a population) of patients associated with biomarker data stored in an aggregated database (e.g., the one or more aggregated databases 295 of FIG. 2). The one or more distinguishing characteristics can include various demographic-related parameters related to: occupation, geographic location, age, gender, ethnicity, income level, family medical history (e.g., a relative diagnosed with a particular autoimmune disorder), and the like.

At block 420, system 100 can populate the subpopulation configuration with a risk assessment age value received from the entity. The risk assessment age can define an age at which risk of developing an autoimmune disorder will be calculated (or determined) using a biomarker dataset for the particular subpopulation defined by the one or more distinguishing characteristics. In an embodiment, system 100 can store the subpopulation configuration populated with the one or more distinguishing characteristics and the risk assessment age in memory (e.g., memory 110). In an embodiment, system 100 can receive the one or more distinguishing characteristics and/or the risk assessment age from the entity via an interface element of a graphical user interface. In an embodiment, system 100 can receive the one or more distinguishing characteristics and/or the risk assessment age from a predefined subpopulation configuration file stored in memory (e.g., memory 110).

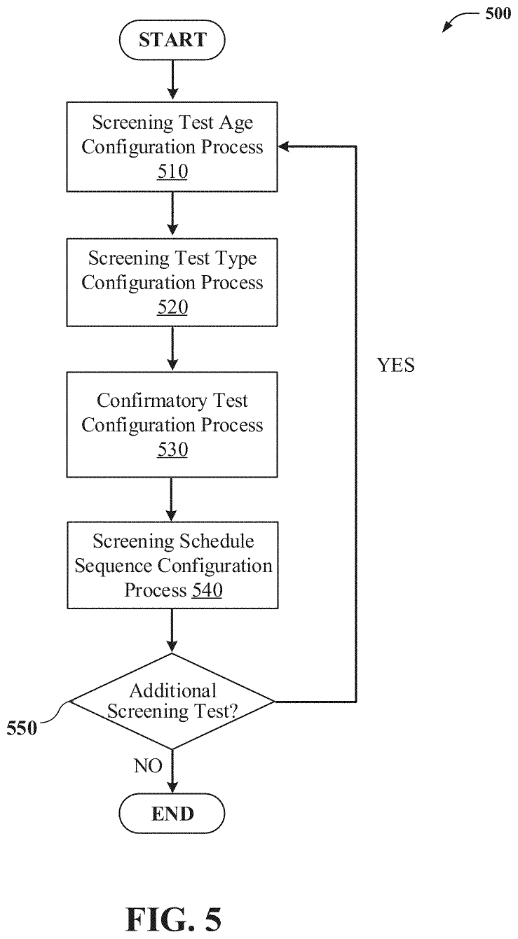

FIG. 5 illustrates an example, non-limiting operational flow for implementing a screening schedule configuration process 500, in accordance with one or more embodiments described herein. In an embodiment, the screening schedule configuration process 230 of autoimmune disorder screening schedule evaluation process 200 can be implemented using screening schedule configuration process 500. In general, a screening schedule configuration can define one or more subsets of biomarker data within a biomarker dataset that evaluation component 150 utilizes to determine performance metrics for a corresponding screening schedule.

At 510, system 100 can populate a screening test configuration of a screening schedule configuration with a testing age for a screening test received from an entity. The testing age can define a landmark age where the screening test is administered in evaluating a corresponding screening schedule. For example, a testing age can define 6-years old as a landmark age where a screening test is administered. In this example, evaluation component 150 could use biomarker data associated with the screening test administered to patients (or subjects) within a subpopulation at an age of 6-years old to evaluate the corresponding screening schedule. In an embodiment, system 100 can populate the screening test configuration with a tolerance range for the testing age received from the entity. In an embodiment, the tolerance range can be defined by an upper limit and a lower limit. For example, a testing age can define 6-years old as a landmark age where a screening test is administered and a tolerance range of the testing age can be defined by an upper limit of 6-years-and-6-months-old and an lower limit of 5-years-and-6-months-old. In this example, evaluation component 150 could use biomarker data associated with the screening test administered to patients (or subjects) within a subpopulation between the ages of 5-years-and-6-months-old and 6-years-and-6-months-old to evaluate the corresponding screening schedule.

At 520, system 100 can populate the screening test configuration with a test type for the screening test received from the entity. The test type can define a particular screening test that is utilized in evaluating the corresponding screening schedule. Example test types include: a single islet autoantibody test; a multiple islet autoantibody test; an IAb count test; an antibody presence test; an antibody absence test; an antibody level test; a disease-specific antibody test (e.g., endomysial antibodies and antibodies to gliadin and reticulin for Celiac Disease); and the like. In an embodiment, the test type for the screening test can be selected from a pre-defined list of testing types. At 530, system 100 can populate the screening test configuration with a confirmatory test option selection for the screening test received from the entity. The confirmatory test option selection can specify whether biomarker data associated with positive screening test results lacking confirmatory tests can be utilized in evaluating the corresponding screening schedule. For example, the confirmatory test option selection can specify that biomarker data associated with positive screening test results lacking confirmatory tests can be utilized in evaluating the corresponding screening schedule. In this example, evaluation component 150 could use biomarker data associated with positive screening test results to evaluate the corresponding screening schedule regardless of whether confirmatory tests were performed. As another example, the confirmatory test option selection can specify that biomarker data associated with positive screening test results lacking confirmatory tests cannot be utilized in evaluating the corresponding screening schedule. In this example, evaluation component 150 could not use biomarker data associated with positive screening test results lacking confirmatory tests to evaluate the corresponding screening schedule.

At 540, system 100 can populate the screening test configuration with a sequence slot value for the screening test received from the entity. The sequence slot value can define a relative position of the screening test within a sequence of screening tests comprising the corresponding screening schedule. For example, a sequence slot value can define the relative position of the screening test as a second screening test administered to patients (or subjects) in a subpopulation within a sequence of screening tests comprising the corresponding screening schedule. In this example, evaluation component 150 could use biomarker data associated with the screening test administered as a second screening test administered to patients (or subjects) in the subpopulation withing the sequence of screening tests to evaluate the corresponding screening schedule. At 550, system 100 can evaluate whether input indicative of an additional screening test configuration is received from the entity. If the evaluation at 550 determines that input indicative of an additional screening test configuration is received, operational flow 500 proceeds to 510. Alternatively, if the evaluation at 550 determines that input indicative of an additional screening test configuration was not received, operational flow 500 terminates.

In an embodiment, system 100 can store the screening schedule configuration populated with one or more screening test configurations in memory (e.g., memory 110). In an embodiment, system 100 can receive the test age, the test type, the confirmatory test value, the sequence slot value, and/or the input indicative of an additional screening test configuration from the entity via an interface element of a graphical user interface. In an embodiment, system 100 can receive the test age, the test type, the confirmatory test value, the sequence slot value, and/or the input indicative of an additional screening test configuration from a predefined screening schedule configuration file stored in memory (e.g., memory 110).

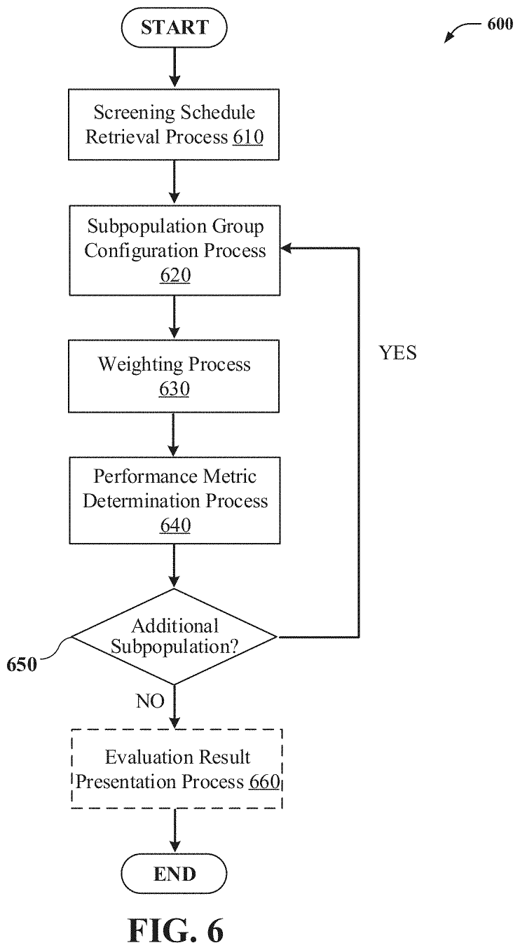

FIG. 6 illustrates an example, non-limiting operational flow for implementing a screening schedule evaluation subprocess 600, in accordance with one or more embodiments described herein. In an embodiment, the screening schedule evaluation subprocess 240 of autoimmune disorder screening schedule evaluation process 200 can be implemented using screening schedule evaluation subprocess 600.

At 610, evaluation component 150 of system 100 can retrieve a screening schedule configuration stored in memory. At 620, pre-processing component 140 can retrieve a subpopulation configuration stored in memory. The pre-processing component 140 can generate a biomarker dataset for a subpopulation defined by the subpopulation configuration. In an embodiment, pre-processing component can generate the biomarker dataset for the subpopulation using the biomarker dataset generation process 320 of aggregated database registration process 300. In an embodiment, the screening schedule configuration and/or the subpopulation configuration is stored in memory 110. In an embodiment, the screening schedule configuration and/or the subpopulation configuration is retrieved via a network interface. At 630, weighting component 160 can assign weights to subject-specific subsets of the biomarker dataset based on longitudinally available data to compensate for irregular data within the one or more aggregated databases 295. In an embodiment, weighting component 160 can assign the weights using an inverse probability weighting mechanism.

At 640, evaluation component 150 can determine a performance metric for the screening schedule based on the biomarker dataset for the subpopulation. At 650, system 100 can evaluate whether input indicative of an additional subpopulation configuration is received from an entity. If the evaluation at 650 determines that input indicative of an additional subpopulation configuration is received, operational flow 600 proceeds to 620. Alternatively, if the evaluation at 650 determines that input indicative of an additional subpopulation configuration was not received, operational flow 600 proceeds to 660. At 660, system 100 can optionally generate a graphical user interface that presents the performance metric for the screening schedule determined using the biomarker dataset for the subpopulation. In an embodiment, the graphical user interface can further present one or more performance metrics for the screening schedule corresponding to an additional subpopulation based on an additional biomarker dataset. In an embodiment, pre-processing component 140 generates the additional biomarker dataset for the additional subpopulation that is distinct from the subpopulation by virtue of a distinguishing characteristic.

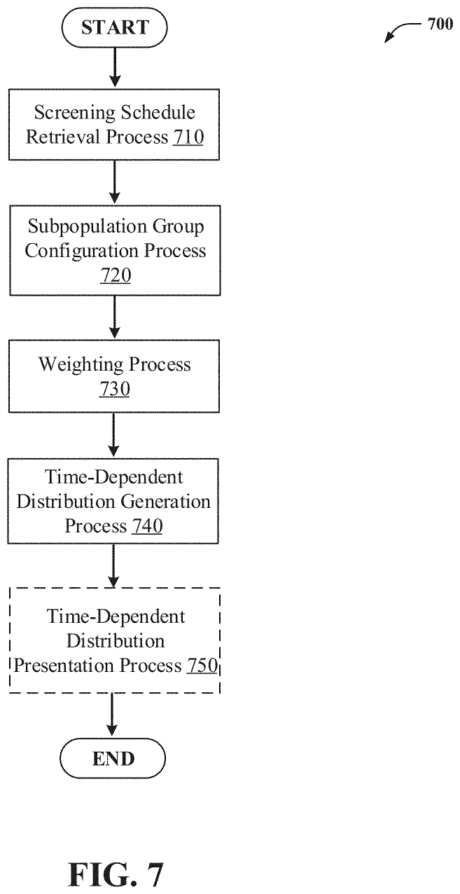

FIG. 7 illustrates an example, non-limiting operational flow for implementing a time-dependent distribution data subprocess 700, in accordance with one or more embodiments described herein. In an embodiment, the time-dependent distribution data subprocess 250 of autoimmune disorder screening schedule evaluation process 200 can be implemented using time-dependent distribution data subprocess 700. At 710, evaluation component 150 of system 100 can retrieve a screening schedule configuration stored in memory. At 720, pre-processing component 140 can retrieve a subpopulation configuration stored in memory. The pre-processing component 140 can generate a biomarker dataset for a subpopulation defined by the subpopulation configuration. In an embodiment, pre-processing component can generate the biomarker dataset for the subpopulation using the biomarker dataset generation process 320 of aggregated database registration process 300. In an embodiment, the screening schedule configuration and/or the subpopulation configuration is stored in memory 110. In an embodiment, the screening schedule configuration and/or the subpopulation configuration is retrieved via a network interface. At 730, weighting component 160 can assign weights to subject-specific subsets of the biomarker dataset based on longitudinally available data to compensate for irregular data within the one or more aggregated databases 295. In an embodiment, weighting component 160 can assign the weights using an inverse probability weighting mechanism.

At 740, evaluation component 150 can generate time-dependent distribution data for a plurality of groups comprising the subpopulation using the screening schedule. In an embodiment, the plurality of groups can include: patients (or subjects) associated with biomarker data associated with positive screening test results; patients associated with biomarker data associated with negative screening test results; patients lacking any biomarker data within the biomarker dataset; or a combination thereof. In an embodiment, the time-dependent distribution data for the plurality of groups comprising the subpopulation include high-level statistical data regarding screening test results associated with the screening schedule. In an embodiment, the high-level statistical data regarding the screening test results can include a distribution of baseline biomarkers (e.g., genetic markers based on Human Leukocyte Antigen (HLA) class II genes) within a respect group in the plurality of groups. In an embodiment, the high-level statistical data regarding the screening test results can include a distribution of disease onset over time within a respect group in the plurality of groups. In an embodiment, the high-level statistical data regarding the screening test results can include a distribution of follow-up over time within a respect group in the plurality of groups. In an embodiment, the high-level statistical data regarding the screening test results can include a distribution of different combinations of biomarkers within a respect group in the plurality of groups. At 750, system 100 can optionally generate a graphical user interface that presents the time-dependent distribution data for the plurality of groups comprising the subpopulation.

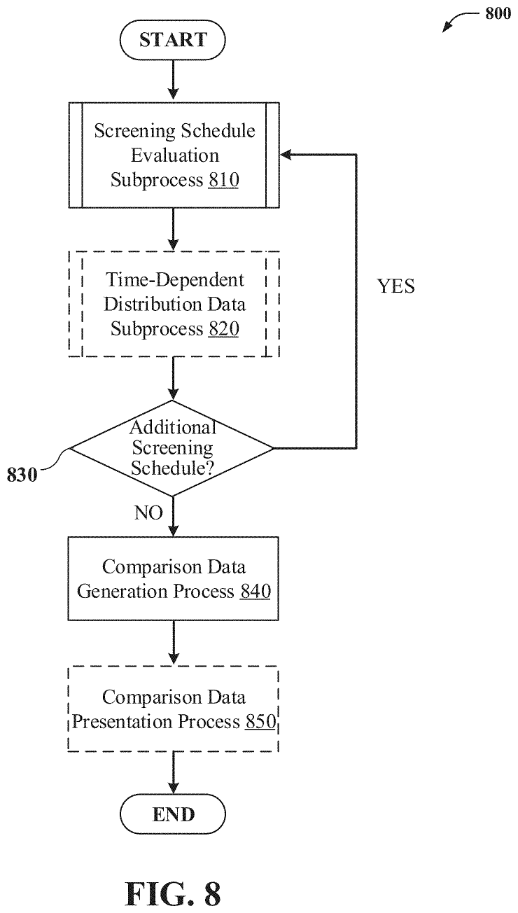

FIG. 8 illustrates an example, non-limiting operational flow for implementing a screening schedule comparison subprocess 800, in accordance with one or more embodiments described herein. In an embodiment, the screening schedule comparison subprocess 260 of autoimmune disorder screening schedule evaluation process 200 can be implemented using screening schedule comparison subprocess 800. At 810, evaluation component 150 of system 100 can execute a screening schedule evaluation subprocess to determine a performance metric for a screening schedule based on a biomarker dataset for a subpopulation. In an embodiment, evaluation component 150 can implement the screening schedule evaluation subprocess utilizing screening schedule evaluation process 600 of FIG. 6. At 820, evaluation component 150 can optionally execute time-dependent distribution data subprocess to generate time-dependent distribution data for the screening schedule based on the biomarker dataset for the subpopulation. In an embodiment, evaluation component 150 can implement the optional time-dependent distribution data subprocess utilizing time-dependent distribution data subprocess 700 of FIG. 7.

At 830, system 100 can evaluate whether input indicative of an additional screening schedule configuration is received from the entity. If the evaluation at 830 determines that input indicative of an additional screening schedule configuration is received, operational flow 800 proceeds to 810. Alternatively, if the evaluation at 830 determines that input indicative of an additional screening schedule configuration was not received, operational flow 800 proceeds to 840. At 840, evaluation component 150 can generate comparison data for multiple screening schedules by analyzing respective performance metrics of the multiple screening schedules determined using the biomarker dataset for the subpopulation. In an embodiment, evaluation component 150 further generates the comparison data by analyzing respective time-dependent distribution data of the multiple screening schedules. At 850, system 100 can optionally generate a graphical user interface that presents the comparison data for multiple screening schedules.

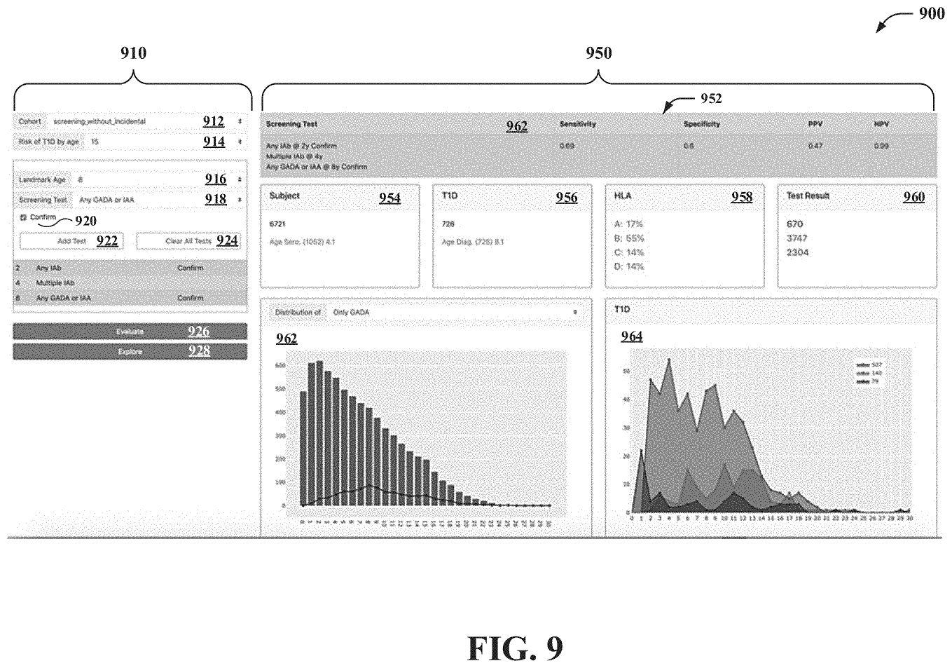

FIG. 9 illustrates an example, non-limiting graphical user interface 900 for facilitating autoimmune disorder screening schedule evaluations, in accordance with one or more embodiments described herein. Graphical user interface 900 includes an input portion 910 via which system 100 can receive information from an entity and an output portion 920 via which system 100 can present information to an entity.

Input portion 910 includes interface elements 912-926. An entity can submit a selection of a subpopulation configuration to system 100 via interface element 912. An entity can submit a risk assessment age value to system 100 via interface element 914. An entity can submit a test age to system 100 via interface element 916. An entity can submit a test type to system 100 via interface element 918. An entity can submit a confirmatory test option selection to system 100 via interface element 920. An entity can submit input indicative of an additional screening test configuration to system 100 via interface element 922. An entity can submit input indicative of screening schedule configuration reset to system via interface element 924. An entity can trigger execution of a screening schedule evaluation subprocess (e.g., screening schedule evaluation subprocesses 240 and 600 of FIGS. 2 and 6, respectively) by system 100 via interface element 926. An entity can trigger execution of a time-dependent distribution data subprocess (e.g., time-dependent distribution data subprocesses 250 and 700 of FIGS. 2 and 7, respectively) by system 100 via interface element 928.

Output portion 950 includes interface elements 952-964. System 100 can present performance metric information for a screening schedule to an entity via interface element 952. System 100 can present a number of subjects and a median age of two consecutive positive biomarkers to an entity via interface element 954. System 100 can present a number of clinically diagnosed subjects and a median age of diagnosis to an entity via interface element 956. System 100 can present a distribution of genetic biomarker subgroups (e.g., high-risk to low-risk HLA groups) to an entity via interface element 958. System 100 can present a number of subjects testing positive, a number of subjects testing negative, and/or a number of subjects that did not perform the screening test due to missing test information to an entity via interface element 960. System 100 can present time-dependent distribution data including a distribution of different combinations of biomarkers within a respect group in the plurality of groups to an entity via interface element 962. System 100 can present time-dependent distribution data including a distribution of disease onset over time within a respect group in a plurality of groups comprising a subpopulation to an entity via interface element 964.

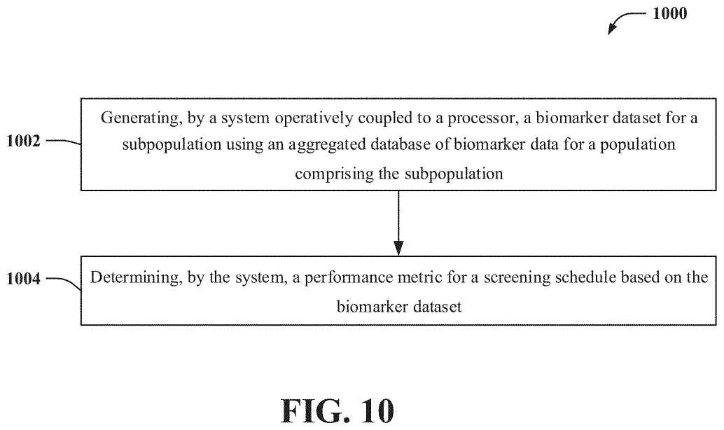

FIG. 10 illustrates a flow diagram of an example, non-limiting computer-implemented method 1000 of facilitating autoimmune disorder screening schedule evaluations, in accordance with one or more embodiments described herein. Repetitive description of like elements employed in other embodiments described herein is omitted for sake of brevity.

At 1002, the computer-implemented method 1000 can comprise generating, by a system operatively coupled to a processor (e.g., with pre-processing component 140), a biomarker dataset for a subpopulation using an aggregated database of biomarker data for a population comprising the subpopulation. In an embodiment, the performance metric can include: a specificity metric, a sensitivity metric, a positive predictive value metric, a negative predictive value metric, or a combination thereof. In an embodiment, the system can generate the biomarker dataset by comparing metadata of the aggregated database with a filtering criterion that defines a distinguishing characteristic of the subpopulation. In an embodiment, the aggregated database can include: electronic health record data, disease registry data, or a combination thereof.

At 1004, the computer-implemented method 1000 can comprise determining, by the system (e.g., with evaluation component 150), a performance metric for a screening schedule based on the biomarker dataset. In an embodiment, the performance metric can quantify an effectiveness of the screening schedule in identifying subjects within the subpopulation that are at risk of developing an autoimmune disorder. In an embodiment, the performance metric can quantify an effectiveness of the screening schedule in identifying subjects at risk for developing an autoimmune disorder in the subpopulation or a similar subpopulation.

In an embodiment, the computer-implemented method 1000 can further comprise: assigning, by the system (e.g., with weighting component 160), weights to subject-specific subsets of the biomarker dataset based on longitudinally available data to compensate for irregular data within the aggregated database. In an embodiment, the computer-implemented method 1000 can further comprise: creating, by the system (e.g., with scheduling component 170), an optimal screening schedule for the subpopulation based on the biomarker dataset. In an embodiment, the computer-implemented method 1000 can further comprise: generating, by the system (e.g., with evaluation component 150), time-dependent distribution data for a plurality of groups comprising the subpopulation using the screening schedule.

In an embodiment, the computer-implemented method 1000 can further comprise: generating, by the system (e.g., with evaluation component 150), comparison data for a plurality of screening schedules by analyzing respective performance metrics of the plurality of screening schedules determined using the biomarker dataset. In an embodiment, the computer-implemented method 1000 can further comprise: generating, by the system (e.g., with pre-processing component 140), an additional biomarker dataset for an additional subpopulation that is distinct from the subpopulation by virtue of a distinguishing characteristic. In an embodiment, the computer-implemented method 1000 can further comprise: determining, by the system (e.g., with evaluation component 150), one or more performance metrics for the screening schedule corresponding to the additional subpopulation based on the additional biomarker dataset.

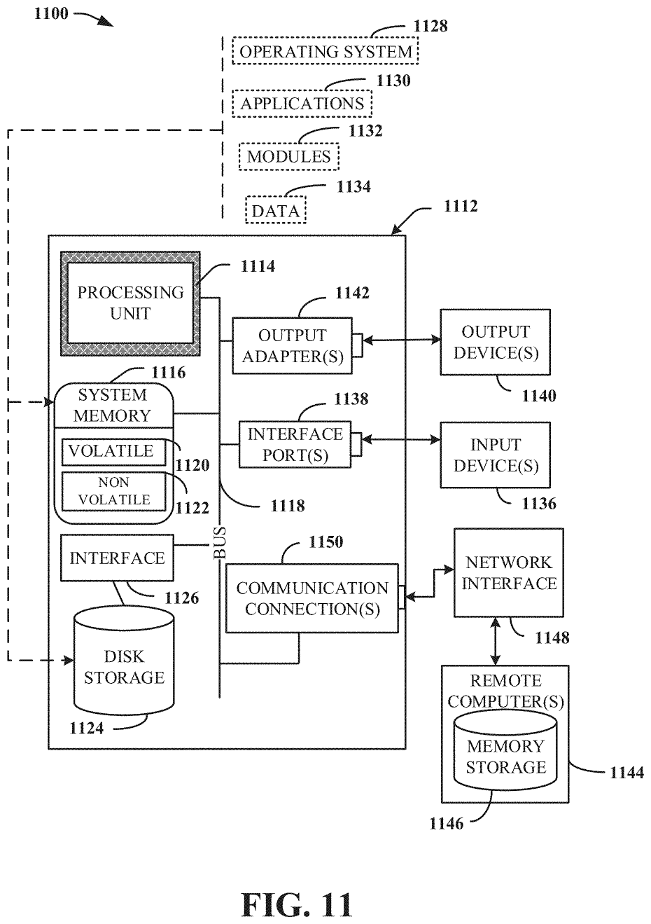

In order to provide a context for the various aspects of the disclosed subject matter, FIG. 11 as well as the following discussion are intended to provide a general description of a suitable environment in which the various aspects of the disclosed subject matter can be implemented. FIG. 11 illustrates a suitable operating environment 1100 for implementing various aspects of this disclosure can also include a computer 1112. The computer 1112 can also include a processing unit 1114, a system memory 1116, and a system bus 1118. The system bus 1118 couples system components including, but not limited to, the system memory 1116 to the processing unit 1114. The processing unit 1114 can be any of various available processors. Dual microprocessors and other multiprocessor architectures also can be employed as the processing unit 1114. The system bus 1118 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1094), and Small Computer Systems Interface (SCSI). The system memory 1116 can also include volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can include read only memory (ROM), programmable ROM (PROM), electrically programmable ROM (EPROM), electrically erasable programmable ROM (EEPROM), flash memory, or nonvolatile random-access memory (RAM) (e.g., ferroelectric RAM (FeRAM). Volatile memory 1120 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1112 can also include removable/non-removable, volatile/non-volatile computer storage media. FIG. 11 illustrates, for example, a disk storage 1124. Disk storage 1124 can also include, but is not limited to, devices like a magnetic disk drive, floppy disk drive, tape drive, Jaz drive, Zip drive, LS-100 drive, flash memory card, or memory stick. The disk storage 1124 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1124 to the system bus 1118, a removable or non-removable interface is typically used, such as interface 1126. FIG. 11 also depicts software that acts as an intermediary between users and the basic computer resources described in the suitable operating environment 1100. Such software can also include, for example, an operating system 1128. Operating system 1128, which can be stored on disk storage 1124, acts to control and allocate resources of the computer 1112. System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems. A user enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port can be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It can be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a computer, a server, a router, a network PC, a workstation, a microprocessor-based appliance, a peer device or other common network node and the like, and typically can also include many or the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1150 refers to the hardware/software employed to connect the network interface 1148 to the system bus 1118. While communication connection 1150 is shown for illustrative clarity inside computer 1112, it can also be external to computer 1112. The hardware/software for connection to the network interface 1148 can also include, for exemplary purposes only, internal and external technologies such as, modems including regular telephone grade modems, cable modems and DSL modems, ISDN adapters, and Ethernet cards.

The present invention may be a system, a method, an apparatus and/or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices. For example, in one or more embodiments, computer executable components can be executed from memory that can include or be comprised of one or more distributed memory units. As used herein, the term “memory” and “memory unit” are interchangeable. Further, one or more embodiments described herein can execute code of the computer executable components in a distributed manner, e.g., multiple processors combining or working cooperatively to execute code from one or more distributed memory units. As used herein, the term “memory” can encompass a single memory or memory unit at one location or multiple memories or memory units at one or more locations.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes the following computer-executable components stored in memory:
a pre-processing component that generates a biomarker dataset for a subpopulation using an aggregated database of biomarker data for a population comprising the subpopulation; and
an evaluation component that determines a performance metric for a screening schedule based on the biomarker dataset, wherein the performance metric quantifies an effectiveness of the screening schedule in identifying subjects within the subpopulation that are at risk of developing an autoimmune disorder.

2. The system of claim 1, wherein the performance metric includes: a specificity metric, a sensitivity metric, a positive predictive value metric, a negative predictive value metric, or a combination thereof.

3. The system of claim 1, wherein the aggregated database includes: electronic health record data, disease registry data, or a combination thereof.

4. The system of claim 1, wherein the pre-processing component generates the biomarker dataset by comparing metadata of the aggregated database with a filtering criterion that defines a distinguishing characteristic of the subpopulation.

5. The system of claim 1, wherein the aggregated database includes biomarker data in a non-standardized format, and wherein the pre-processing component generates the biomarker dataset by converting the biomarker data in the non-standardized format into a standardized format of the biomarker dataset.

6. The system of claim 1, further comprising:
a weighting component that assigns weights to subject-specific subsets of the biomarker dataset based on longitudinally available data to compensate for irregular data within the aggregated database.

7. The system of claim 1, further comprising:
a scheduling component that creates an optimal screening schedule for the subpopulation based on the biomarker dataset.

8. The system of claim 1, wherein the evaluation component further generates time-dependent distribution data for a plurality of groups comprising the subpopulation using the screening schedule.

9. The system of claim 1, wherein the evaluation component further generates comparison data for a plurality of screening schedules by analyzing respective performance metrics of the plurality of screening schedules determined using the biomarker dataset.

10. The system of claim 1, wherein the pre-processing component further generates an additional biomarker dataset for an additional subpopulation that is distinct from the subpopulation by virtue of a distinguishing characteristic, and wherein the evaluation component further determines one or more performance metrics for the screening schedule corresponding to the additional subpopulation based on the additional biomarker dataset.

11. A computer-implemented method, comprising:
generating, by a system operatively coupled to a processor, a biomarker dataset for a subpopulation using an aggregated database of biomarker data for a population comprising the subpopulation; and
determining, by the system, a performance metric for a screening schedule based on the biomarker dataset, wherein the performance metric quantifies an effectiveness of the screening schedule in identifying subjects within the subpopulation that are at risk of developing an autoimmune disorder.

12. The computer-implemented method of claim 11, wherein the performance metric includes: a specificity metric, a sensitivity metric, a positive predictive value metric, a negative predictive value metric, or a combination thereof.

13. The computer-implemented method of claim 11, wherein the system generates the biomarker dataset by comparing metadata of the aggregated database with a filtering criterion that defines a distinguishing characteristic of the subpopulation.

14. The computer-implemented method of claim 11, further comprising:
assigning, by the system, weights to subject-specific subsets of the biomarker dataset based on longitudinally available data to compensate for irregular data within the aggregated database.

15. The computer-implemented method of claim 11, further comprising:
creating, by the system, an optimal screening schedule for the subpopulation based on the biomarker dataset.

16. The computer-implemented method of claim 11, further comprising:
generating, by the system, time-dependent distribution data for a plurality of groups comprising the subpopulation using the screening schedule.

17. The computer-implemented method of claim 11, further comprising:
generating, by the system, comparison data for a plurality of screening schedules by analyzing respective performance metrics of the plurality of screening schedules determined using the biomarker dataset.

18. The computer-implemented method of claim 11, further comprising:
generating, by the system, an additional biomarker dataset for an additional subpopulation that is distinct from the subpopulation by virtue of a distinguishing characteristic; and
determining, by the system, one or more performance metrics for the screening schedule corresponding to the additional subpopulation based on the additional biomarker dataset.

19. A computer program product comprising a computer readable storage medium having program instructions embodied therewith, the program instructions executable by a processor to cause the processor to:
generate, by the processor, a biomarker dataset for a subpopulation using an aggregated database of biomarker data for a population comprising the subpopulation; and
determine, by the processor, a performance metric for a screening schedule based on the biomarker dataset, wherein the performance metric quantifies an effectiveness of the screening schedule in identifying subjects within the subpopulation that are at risk of developing an autoimmune disorder.

20. The computer program product of claim 19, the program instructions executable by the processor to further cause the processor to:
assign, by the processor, weights to subject-specific subsets of the biomarker dataset based on longitudinally available data to compensate for irregular data within the aggregated database.
